(19) United States
(12) Patent Application Publication
(10) Pub. No.: US 2019/0357878 A1
(43) Pub. Date: Nov. 28, 2019

(54) ULTRASOUND OBSERVATION DEVICE AND OPERATION METHOD OF ULTRASOUND OBSERVATION DEVICE

(71) Applicant: OLYMPUS CORPORATION, Tokyo (JP)

(72) Inventor: Kazuhito NEMOTO, Tokyo (JP)

(73) Assignee: OLYMPUS CORPORATION, Tokyo (JP)

(21) Appl. No.: 16/532,724

(22) Filed: Aug. 6, 2019

Related U.S. Application Data
(63) Continuation of application No. PCT/JP2018/006256, filed on Feb. 21, 2018.

(30) Foreign Application Priority Data
Mar. 7, 2017 (JP) 2017-042554

(51) Int. Cl. A61B 8/12; A61B 8/00

(52) U.S. Cl. CPC A61B 8/12; A61B 8/445; A61B 1/018; A61B 8/4461; A61B 8/4483

(57) ABSTRACT

An ultrasound observation device is configured to generate, based on ultrasound signals acquired by an ultrasound transducer performing transmission and reception to and from an observation target, plural ultrasound images along time series. The ultrasound observation device includes: a movement amount calculating circuit configured to calculate, from the plural ultrasound images, an amount of movement that is an amount moved by a subject captured in an ultrasound image of the latest frame relatively to the subject captured in an ultrasound image of a past frame; a reliability determining circuit configured to determine a reliability of the amount of movement calculated by the movement amount calculating circuit; and a frozen image selecting circuit configured to select a frozen image from the plural ultrasound images, based on the amount of movement and the reliability, when input of a freeze instruction signal has been received.

### CROSS-REFERENCE TO RELATED APPLICATION

This application is a continuation of PCT International Application No. PCT/JP2018/006256 filed on Feb. 21, 2018, which designates the United States, incorporated herein by reference, and which claims the benefit of priority from Japanese Patent Application No. 2017-042554, filed on Mar. 7, 2017, incorporated herein by reference.

### BACKGROUND

### 1. Technical Field

The present disclosure relates to an ultrasound observation device and an operation method of the ultrasound observation device.

### 2. Related Art

In the related art, there is an ultrasound observation device, which generates ultrasound images that are scanned images of cross sections of a subject, based on ultrasound signals acquired by an ultrasound transducer performing transmission to and reception from an observation target, the ultrasound transducer having been arranged at a distal end of an insertion portion to be inserted into the subject. Known as this ultrasound observation device is a device having a freezing function of specifying and displaying an image of a desired time point while a display device is displaying subject images live. Furthermore, also known is a device having a prefreezing function of always temporarily storing subject images of a certain time period beforehand, and selecting a frozen image from the temporarily stored images at a time point when a user pushes a freezing switch, the frozen image being an image having less blurring caused by relative movement (see, for example, Japanese Laid-open Patent Publication No. 2004-024559).

Known as a technique for selecting an image with less movement by this prefreezing function is an ultrasound observation device that selects a frozen image, based on movement information in ultrasound images (see, for example, Japanese Laid-open Patent Publication No. 2015-131100). According to this technique, at the time of capturing of the ultrasound images, amounts of movement of the ultrasound transducer in a direction parallel to the scan surface represented by the ultrasound images are calculated from the ultrasound images, and an image with the smallest amount of movement is selected as the frozen image.

According to an embodiment of the disclosure, an ultrasound observation device enabling appropriate selection of an image with less movement upon image selection by a prefreezing function, an operation method of the ultrasound observation device, and an operation program of the ultrasound observation device are able to be realized.

### SUMMARY

In some embodiments, provided is an ultrasound observation device configured to generate, based on ultrasound signals acquired by an ultrasound transducer performing transmission and reception to and from an observation target, plural ultrasound images along time series. The ultrasound observation device includes: a movement amount calculating circuit configured to calculate, from the plural ultrasound images, an amount of movement that is an amount moved by a subject captured in an ultrasound image of the latest frame relatively to the subject captured in an ultrasound image of a past frame; a reliability determining circuit configured to determine a reliability of the amount of movement calculated by the movement amount calculating circuit; and a frozen image selecting circuit configured to select a frozen image from the plural ultrasound images, based on the amount of movement and the reliability, when input of a freeze instruction signal has been received.

In some embodiments, provided is an operation method of an ultrasound observation device configured to generate, based on ultrasound signals acquired by an ultrasound transducer performing transmission and reception to and from an observation target, plural ultrasound images along time series. The operation method includes: calculating, from the plural ultrasound images, an amount of movement that is an amount moved by a subject captured in an ultrasound image of the latest frame relatively to the subject captured in an ultrasound image of a past frame; determining a reliability of the calculated amount of movement; and selecting a frozen image from the plural ultrasound images, based on the amount of movement and the reliability, when input of a freeze instruction signal has been received.

The above and other features, advantages and technical and industrial significance of this invention will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating a configuration of an ultrasound diagnosis system including an ultrasound observation device according to a first embodiment of the disclosure;

FIG. 2 is a diagram illustrating measured areas (blocks) set in an image for calculation of amounts of movement;

FIG. 3 is a diagram illustrating how an amount of movement is calculated;

FIG. 4 is a diagram illustrating how areas are set in an ultrasound image; and

FIG. 5 is a block diagram illustrating a configuration of an ultrasound diagnosis system including an ultrasound observation device according to a reference example of the disclosure.

### DETAILED DESCRIPTION

Hereinafter, embodiments of an ultrasound observation device, an operation method of the ultrasound observation device, and an operation program of the ultrasound observation device, according to the disclosure will be described by reference to the drawings. The disclosure is not limited by these embodiments. The disclosure is applicable generally to an ultrasound observation device having a prefreezing function, an operation method of the ultrasound observation device, and an operation program of the ultrasound observation device.

Furthermore, any elements that are the same or corresponding to each other are assigned with the same reference sign throughout the drawings, as appropriate. Moreover, it needs to be noted that the drawings are schematic, and relations among dimensions of each element, proportions among elements, and the like may be different from the actual ones. Portions having different dimensional relations and proportions among the drawings may also be included.

### First Embodiment

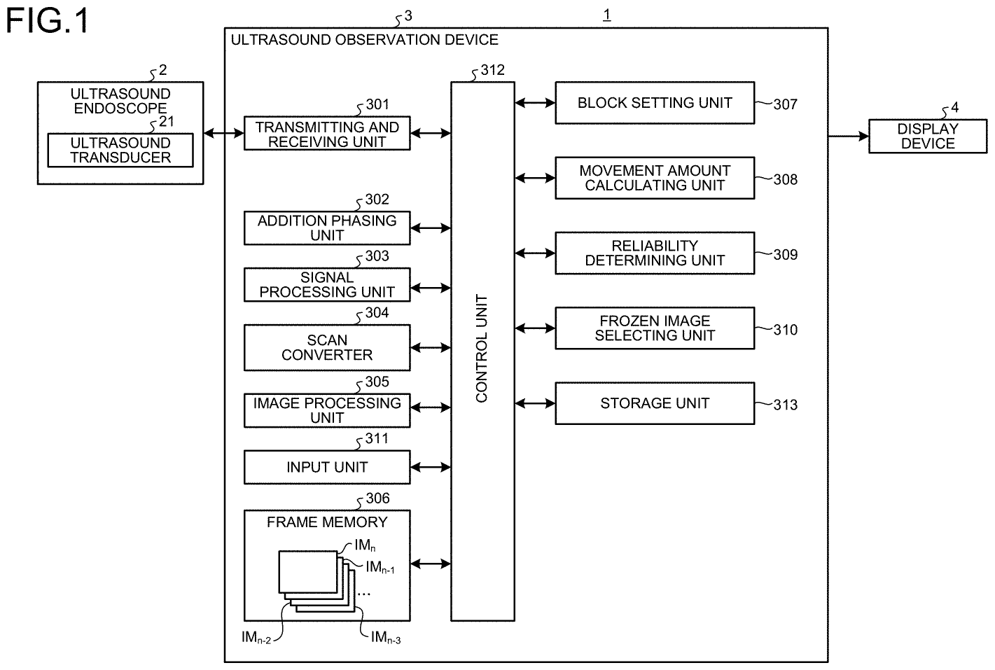

FIG. 1 is a block diagram illustrating a configuration of an ultrasound diagnosis system including an ultrasound observation device according to a first embodiment of the disclosure. An ultrasound diagnosis system 1 illustrated in FIG. 1 includes: an ultrasound endoscope 2, which transmits ultrasound to a subject that is an observation target, and which receives the ultrasound reflected by the subject; an ultrasound observation device 3 that generates an ultrasound image based on an ultrasound signal acquired by the ultrasound endoscope 2; and a display device 4 that displays the ultrasound image generated by the ultrasound observation device 3.

The ultrasound endoscope 2 has, at a distal end portion thereof, an ultrasound transducer 21 that: converts an electric pulse signal received from the ultrasound observation device 3, into ultrasound pulses (acoustic pulses); emits the ultrasound pulses to the subject; converts ultrasound echoes reflected by the subject, into an electric echo signal representing the ultrasound echoes by change in voltage; and outputs the electric echo signal. The ultrasound transducer 21 is arranged at a distal end of an insertion portion to be inserted into the subject. The ultrasound transducer 21 is of the convex type or the linear type, such that a scan surface of the ultrasound transducer 21 becomes parallel to an axial direction of the distal end of the insertion portion of the endoscope. In the ultrasound endoscope 2: the ultrasound transducer 21 may be made to perform scanning mechanically; or plural elements may be provided in an array as the ultrasound transducer 21, and made to perform scanning electronically by: electronic switch-over among elements related to transmission and reception; or insertion of delay in transmission and reception by the elements.

Normally, the ultrasound endoscope 2: has an imaging optical system and an imaging element; is inserted in the gastrointestinal tract (the esophagus, the stomach, the duodenum, and the large intestine) or the respiratory organs (the trachea and the bronchus) of the subject; and is able to capture images of the gastrointestinal tract or respiratory organs, and the organs (the pancreas, the gallbladder, the bile duct, the biliary tract, the lymph nodes, the mediastinal organ, the blood vessels, and/or the like) surrounding the gastrointestinal tract or respiratory organs. Furthermore, the ultrasound endoscope 2 has a light guide that guides illumination light to be emitted to the subject at the time of image capturing. This light guide has a distal end portion that reaches the distal end of the insertion portion to be inserted into the subject of the ultrasound endoscope 2, and a proximal end portion connected to a light source device that generates the illumination light.

The ultrasound observation device 3 has a transmitting and receiving unit 301, an addition phasing unit 302, a signal processing unit 303, a scan converter 304, an image processing unit 305, a frame memory 306, a block setting unit 307, a movement amount calculating unit 308, a reliability determining unit 309, a frozen image selecting unit 310, an input unit 311, a control unit 312, and a storage unit 313.

The transmitting and receiving unit 301: is electrically connected to the ultrasound endoscope 2; transmits, based on a predetermined waveform and transmission timing, a transmission signal (a pulse signal) formed of high voltage pulses, to the ultrasound transducer 21; and receives an echo signal that is an electric reception signal, from the ultrasound transducer 21.

A frequency band of the pulse signal transmitted by the transmitting and receiving unit 301 is preferably a wide band substantially covering a linear response frequency band for electric-acoustic conversion of the pulse signal by the ultrasound transducer 21 into ultrasound pulses.

The transmitting and receiving unit 301 also has a function of: transmitting various control signals output by the control unit 312, to the ultrasound endoscope 2; and receiving various types of information including an ID for identification, from the ultrasound endoscope 2, and transmitting the various types of information, to the control unit 312.

The addition phasing unit 302 receives an echo signal from the transmitting and receiving unit 301, and generates and outputs data (hereinafter, referred to as RF data) on a digital radio frequency (RF) signal. The addition phasing unit 302: performs sensitivity time control (STC) correction where amplification is performed with a higher amplification factor for an echo signal larger in reception depth; performs processing, such as filtering, on the amplified echo signal; thereafter generates time domain RF data by A/D conversion of the amplified echo signal processed; and outputs the generated RF data, to the signal processing unit 303. If the ultrasound endoscope 2 has a configuration where the ultrasound transducer 21 having plural elements provided in an array is caused to perform electronic scanning, the addition phasing unit 302 has a multi-channel circuit for beam combination corresponding to the plural elements.

The signal processing unit 303 generates, based on the RF data received from the transmitting and receiving unit 301, digital B-mode reception data. The signal processing unit 303 performs known processing, such as bandpass filtering, envelope demodulation, and logarithmic transformation, on the RF data, and generates the digital B-mode reception data. In the logarithmic transformation, a common logarithm of a quantity resulting from division of the RF data by a reference voltage Vc is expressed as a decibel value. The signal processing unit 303 outputs the generated B-mode reception data, to the image processing unit 305. The signal processing unit 303 is realized by use of a central processing unit (CPU) or various arithmetic operation circuits.

The scan converter 304 converts the scan direction of the B-mode reception data received from the signal processing unit 303, and generates frame data. Specifically, the scan converter 304 converts the scan direction of the B-mode reception data into a display direction of the display device 4 from the scan direction of the ultrasound.

The image processing unit 305 generates B-mode image data (hereinafter, simply referred to as image data also) including an ultrasound image that is a B-mode image displaying thereon amplitude of an echo signal, the amplitude having been converted into luminance. The image processing unit 305 generates the B-mode image data by performing signal processing where known techniques such as gain processing and contrast processing are used for the frame data from the scan converter 304, and performing thinning or the like of data according to a data step width determined according to an image display range in the display device 4. A B-mode image is a gray scale image where red (R), green (G), and blue (B) values, which are variables when the RGB colorimetric system is adopted as a color space, have been made the same.

After performing coordinate transformation where the B-mode reception data from the signal processing unit 303 are rearranged so as to enable correct spatial representation of the scan range, the image processing unit 305 fills in gaps among the B-mode reception data by performing interpolation processing among the B-mode reception data, and generates the B-mode image data.

The frame memory 306 is realized by use of, for example, a ring buffer, and stores therein a certain amount of ultrasound images (a predetermined number of frames N) generated by the image processing unit 305, along time series. When the capacity becomes insufficient (when a predetermined number of frames of B-mode image data have been stored), the predetermined number of frames of the latest ultrasound images are stored in time series order by the oldest B-mode image data being overwritten by the latest B-mode image data. As illustrated in FIG. 1, the frame memory 306 stores therein plural ultrasound images (IMₙ₋₁, IMₙ₋₂, IMₙ₋₃, and so on) that are within the predetermined number of past frames from an ultrasound image IMₙ of the n-th frame (where n is a natural number equal to or greater than 2) that is the latest ultrasound image. Furthermore, the frame memory 306 stores therein amounts of movement calculated by the movement amount calculating unit 308 described later and reliabilities thereof calculated by the reliability determining unit 309 described later, in association with the ultrasound images.

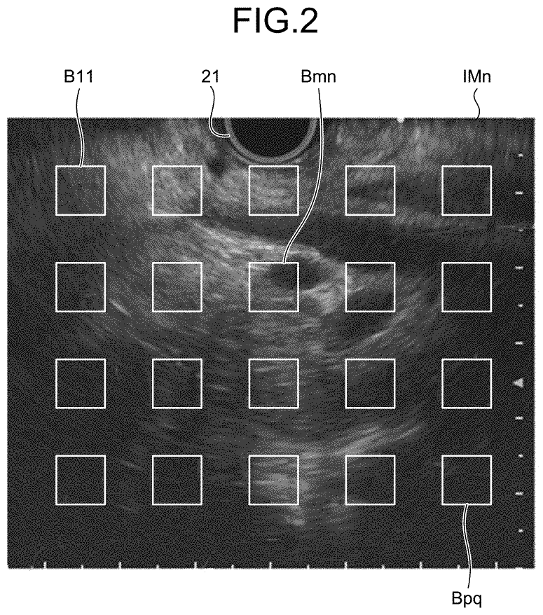

FIG. 2 is a diagram illustrating measured areas (blocks) set in an image for calculation of amounts of movement. As illustrated in FIG. 2, the block setting unit 307 sets vertically “p”×horizontally “q” (where “p” and “q” are natural numbers equal to or greater than 2) blocks from a block B11 to a block Bpq in a lattice shape on an ultrasound image IMₙ that is the latest ultrasound image. The block setting unit 307 is realized by use of a CPU, various arithmetic operation circuits, or the like.

The movement amount calculating unit 308 calculates an amount of movement that is an amount by which a subject captured in the ultrasound image IMₙ of the latest frame has a moved relatively to the subject captured in an ultrasound image of a past frame, from the plural ultrasound images stored in the frame memory 306. Specifically, the movement amount calculating unit 308 calculates the amount of movement by, for example, a known block matching method where the sum of absolute differences (SAD) of pixel values is used, the SAD being a kind of correlation values. The movement amount calculating unit 308 is realized by use of a CPU, various arithmetic operation circuits, or the like.

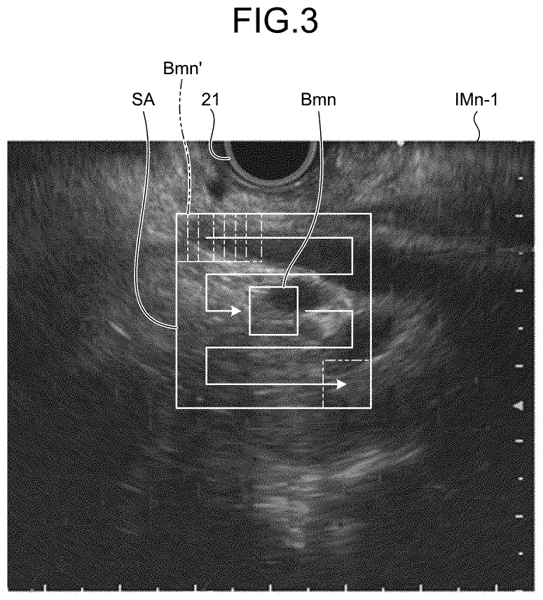

FIG. 3 is a diagram illustrating how the amount of movement is calculated. FIG. 3 is a diagram illustrating how an amount of movement of a block Bmn in FIG. 2 is calculated. As illustrated in FIG. 3, the movement amount calculating unit 308 sequentially sets, along arrows illustrated in FIG. 3, blocks Bmn′ included in a search area SA set around a block Bmn, which is at the same position as the block Bmn set in the ultrasound image IMₙand is set in an immediately preceding ultrasound image IMₙ₋₁, and sequentially calculates correlation values SADs representing similarities between the set blocks Bmn′ and the block Bmn. When the movement amount calculating unit 308 calculates correlation values SADs of all of the blocks included in the search area SA, the movement amount calculating unit 308 calculates, as an amount of movement, a vector from the center of the block Bmn to the center of a block having the smallest correlation value SAD. The movement amount calculating unit 308 repeats this calculation of the amount of movement for each block, and calculates amounts of movement for all of the blocks in the ultrasound image IMₙ. Although an SAD is used herein as a correlation value in the block matching method, any other correlation value (for example, the sum of squared differences (SSD)) may be used. Lastly, the movement amount calculating unit 308 calculates a frame movement amount representing movement of the whole image from a statistical value (the average, the mode, or the like) of the amounts of movement of the plural blocks. Furthermore, the frame movement amount may be calculated by use of only the amounts of movement of blocks high in later described reliability of their amounts of movement.

The reliability determining unit 309 determines the reliability of an amount of movement calculated by the movement amount calculating unit 308. Various methods have been known for determination of reliability representing correctness of a result of block matching, but described herein is a method where a characteristic of a distribution of correlation values SADs is used based on continuity of pixel values in an image, the characteristic being that the distribution approximates a paraboloid near the minimum position. Firstly, the reliability determining unit 309 calculates, as a curved surface, an elliptic parabolic function expressed by Equation (1) below, the curved surface resulting from approximation of correlation values SADs of blocks by the least-square method, the blocks being near the minimum position of the correlation values in the search area SA, the correlation values SADs having been calculated by the movement amount calculating unit 308.

SAD(x, y)=(x−xc)²/a²+(y−yc)²/b²+c (1)

If the distribution of the correlation values SADs is similar to an elliptic paraboloid, the reliability determining unit 309 determines that the reliability of the amount of movement is high. In other words, if the residual error between the actual correlation values SADs and the calculated elliptic parabolic function is less that a predetermined threshold, the reliability determining unit 309 determines that the reliability is high, and if the residual error is equal to or larger than the predetermined threshold, the reliability determining unit 309 determines that the reliability is low. This is because if an area similar to the block Bmn on the IMₙ is not present in the search area SA on IMₙ₋₁, the correlation values SADs will not be distributed paraboloidally. Furthermore, if coefficients “a” and “b” of the elliptic parabolic function are larger than predetermined thresholds, the area is low in contrast and unsuitable for block matching, and thus the reliability may be determined to be low. Moreover, if the coordinates (xc, yc) are outside the search area SA, the destination is presumed to be outside the search area SA and is also unsuitable, and the reliability then may thus be determined to be low.

The method of determining the reliability of an amount of movement is not limited to the above described method, and any other known method, in which the reliability is determined to be low when there are no similar areas between the images, may be used. The reliability determining unit 309 performs this reliability determination for all of the blocks in the ultrasound image IMₙ, and determines reliabilities of the amounts of movement of all of the blocks included in the ultrasound image IMₙ. The reliability determining unit 309 is realized by use of a CPU, various arithmetic operation circuits, or the like.

Movement of the ultrasound transducer 21 in the ultrasound endoscope 2 at the time of observation by use of the ultrasound endoscope 2 includes: a component due to movement (hereinafter, also referred to as parallel movement) along a direction parallel to the scan surface of the ultrasound transducer 21; a component due to movement (hereinafter, also referred to as orthogonal movement) in a direction orthogonal to the scan surface; and a component due to rotation about a rotational axis intersecting the scan surface. A frame movement amount that the movement amount calculating unit 308 is able to calculate by the block matching method is an amount of the component due to the parallel movement of the ultrasound transducer 21.

The reliability calculated by the reliability determining unit 309 is an index determining whether or not the ultrasound transducer 21 includes the components due to the movement other than the parallel movement (the orthogonal movement and rotation). This is because inclusion of the components due to the movement other than the parallel movement in the movement of the ultrasound transducer 21 means that temporally consecutive frames are scanning different cross sections of a subject, and the number of blocks determined to be low in reliability will thus be increased.

When the input unit 311 receives input of a freeze instruction signal, the frozen image selecting unit 310 selects, based on frame movement amounts and reliabilities thereof, a frozen image from the plural ultrasound images stored in the frame memory 306. Specifically, the frozen image selecting unit 310: evaluates each ultrasound image by using an evaluation function having, as variables, frame movement amount of the ultrasound image and proportion of blocks high in reliability of their amounts of movement; and selects, as a frozen image, an ultrasound image that has been evaluated most highly. In other words, the frozen image selecting unit 310 selects a frozen image based on information related to: the parallel movement of the ultrasound transducer 21 evaluated according to the amounts of movement; and the movement other than the parallel movement of the ultrasound transducer 21 evaluated according to the reliabilities. The frozen image selecting unit 310 is realized by use of a CPU, various arithmetic operation circuits, or the like.

The input unit 311 is realized by use of a user interface, such as a keyboard, a mouse, a touch panel, and/or the like, and receives input of various types of information. The input unit 311 receives input of a freeze instruction signal that is input of an instruction causing the display device 4 to display a frozen image by means of a prefreezing function.

The control unit 312 controls the whole ultrasound diagnosis system 1. The control unit 312 is realized by use of a CPU, various arithmetic operation circuits, or the like, that has/have arithmetic operation and control functions. The control unit 312 integrally controls the ultrasound observation device 3 by: reading information recorded and stored in the storage unit 313 from the storage unit 313; and executing various types of arithmetic operation processing related to an operation method of the ultrasound observation device 3. The control unit 312 may be configured by use of the CPU or the like shared with the signal processing unit 303, the block setting unit 307, the movement amount calculating unit 308, the reliability determining unit 309, and the frozen image selecting unit 310.

The storage unit 313 stores therein various programs including an operation program for execution of the operation method of the ultrasound observation device 3. The operation program may be widely distributed by being recorded in a computer readable recording medium, such as a hard disk, a flash memory, a CD-ROM, a DVD-ROM, or a flexible disk. The above described various programs may be acquired by being downloaded via a communication network. The communication network herein is realized by, for example, any existing public network, a local area network (LAN), or a wide area network (WAN), and may be wired or wireless.

The storage unit 313 having the above described configuration is realized by use of: a read only memory (ROM) having the various programs and the like preinstalled therein; a random access memory (RAM) storing therein arithmetic operation parameters, data, and the like for various types of processing; and the like.

According to the first embodiment described above, the frozen image selecting unit 310 selects a frozen image based on frame movement amounts and reliabilities thereof. As a result, the frozen image selecting unit 310 is able to select, as a frozen image, an image that is: small in the amount of movement; small in the parallel movement of the ultrasound transducer 21; and small in the movement other than the parallel movement of the ultrasound transducer 21, the image including many amounts of movement high in reliability. Therefore, the ultrasound observation device 3 is an ultrasound observation device with improved accuracy for selection of an image with less movement upon image selection by means of the prefreezing function.

Frame correlation images each formed of a combination of ultrasound images arranged in time series may be generated and stored along the time series in the frame memory 306, instead of ultrasound images. The frame correlation image is generated by averaging of the ultrasound images arranged in the time series, the averaging including weighting associated with the time series. In this case, the movement amount calculating unit 308 and the reliability determining unit 309 respectively calculate amounts of movement of frame correlation images and reliabilities thereof, and the frozen image selecting unit 310 selects a frozen image from the frame correlation images.

Furthermore, with respect to the first embodiment, the example where the ultrasound transducer 21 is of the convex type or the linear type has been described, but the ultrasound transducer 21 may be of the radial type where the ultrasound scan surface is vertical to the distal end of the insertion portion of the ultrasound endoscope 2. When the ultrasound transducer 21 is of the radial type, the amount of movement calculated by the movement amount calculating unit 308 is the amount of movement in each direction orthogonal to the insertion portion, and the reliability calculated by the reliability determining unit 309 is an index indicating whether or not movement in the direction along the insertion portion and rotation about the rotational axis intersecting the scan surface are included. By the frozen image selecting unit 310 selecting a frozen image by using an evaluation function having, as variables, the amount of movement and the reliability, an image with less movement is able to be selected appropriately as a frozen image, like with the convex type or the linear type.

### Second Embodiment

Processing in an ultrasound observation device according to a second embodiment is different from that according to the first embodiment; and a configuration of the ultrasound observation device according to the second embodiment is the same as that according to the first embodiment, and thus description thereof will be omitted as appropriate.

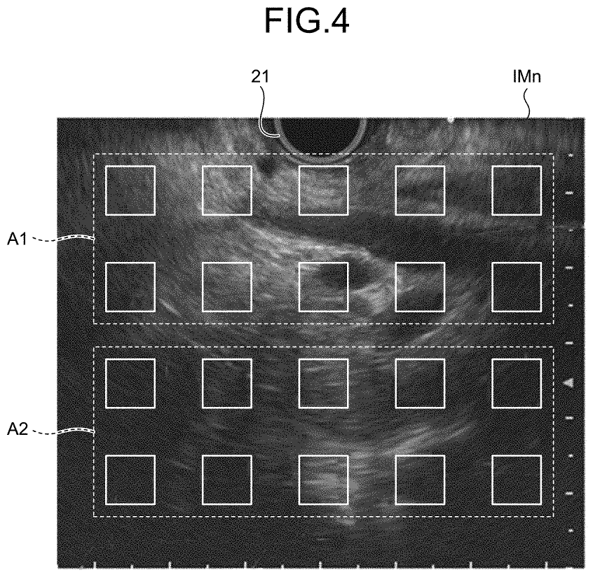

FIG. 4 is a diagram illustrating how areas are set in an ultrasound image. As illustrated in FIG. 4, the frozen image selecting unit 310 divides the ultrasound image IMₙ into an area A1 near the ultrasound transducer 21 and an area A2 more distant than the area A1 from the ultrasound transducer 21. The frozen image selecting unit 310 calculates a proportion of blocks high in reliability of their amounts of movement in each of the area A1 and the area A2. Furthermore, the frozen image selecting unit 310 evaluates each ultrasound image by using an evaluation function having, as variables, frame movement amount of the ultrasound image and proportion of blocks high in reliability of their amounts of movement in each of the area A1 and area A2, and selects, as a frozen image, an ultrasound image that has been evaluated most highly.

When the ultrasound transducer 21 of the convex type or the linear type rotates about an axis that is a distal end portion of the insertion portion, the area A2 that is more distant from the ultrasound transducer 21 in the ultrasound image IMₙ tends to be influenced by the rotation, that is, different areas of the subject will be scanned for the area A2, and thus the reliability of the amounts of movement tends to be reduced. In other words, reliability in the area A2 tends to be reduced more than that in the area A1. Therefore, according to the second embodiment, because the proportion of blocks high in reliability of their amounts of movement different for each area is substituted into the evaluation function used upon selection of a frozen image by the frozen image selecting unit 310, an image with less movement due to rotation about the axis that is the distal end portion of the insertion portion is able to be selected appropriately as a frozen image.

As described above, the frozen image selecting unit 310 may select a frozen image, based on reliability distributions in ultrasound images.

Furthermore, the frozen image selecting unit 310 may select a frozen image by using only the reliability in the area A2. That is, the frozen image selecting unit 310 may select, as a frozen image, an ultrasound image high in reliability in an area (an area distant from the ultrasound transducer 21) where the depth of the observation target relative to the ultrasound transducer 21 is large.

Furthermore, when the ultrasound transducer 21 is of the convex type, the area A1 near the ultrasound transducer 21 and the area A2 more distant than the area A1 from the ultrasound transducer 21 are an upper area and a lower area in an ultrasound image, respectively. In contrast, when the ultrasound transducer 21 is of the radial type, the area A1 near the ultrasound transducer 21 and the area A2 more distant than the area A1 from the ultrasound transducer 21 may be an area near the ultrasound transducer 21 and extending concentrically, and an area around that area and extending concentrically therewith, respectively.

### Third Embodiment

Processing in an ultrasound observation device according to a third embodiment is different from that according to the first embodiment; and a configuration of the ultrasound observation device according to the third embodiment is the same as that of the first embodiment, and description thereof will thus be omitted as appropriate.

Similarly to the second embodiment, the frozen image selecting unit 310 divides the ultrasound image IMₙ into the area A1 near the ultrasound transducer 21 and the area A2 more distant than the area A2 from the ultrasound transducer 21. The frozen image selecting unit 310 then calculates a proportion of blocks high in reliability of their amounts of movement, in each of the area A1 and the area A2. Furthermore, the frozen image selecting unit 310 evaluates each ultrasound image by using an evaluation function having, as variables, frame movement amount of the ultrasound image and proportion of blocks high in reliability of their amounts of movement in each of the whole image, the area A1, and the area A2; and selects, as a frozen image, an ultrasound image that has been evaluated most highly. An image with less movement is thereby able to be selected more appropriately as a frozen image.

### REFERENCE EXAMPLE

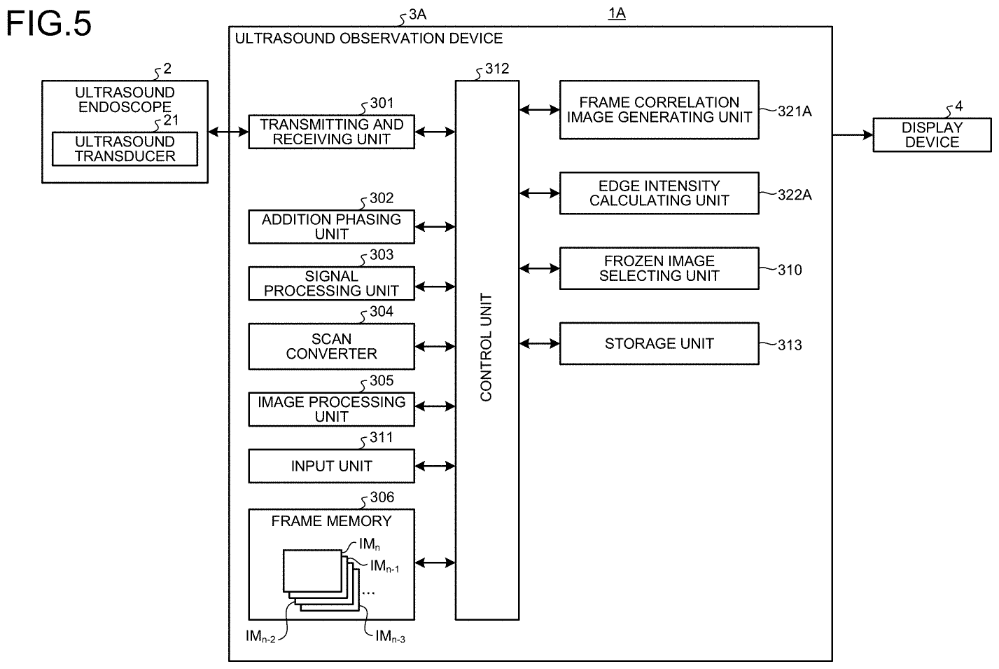

FIG. 5 is a block diagram illustrating a configuration of an ultrasound diagnosis system including an ultrasound observation device according to a reference example of the disclosure. An ultrasound diagnosis system 1A illustrated in FIG. 5 includes an ultrasound observation device 3A having a frame correlation image generating unit 321A and an edge intensity calculating unit 322A.

The frame correlation image generating unit 321A generates a frame correlation image formed of a combination of ultrasound images arranged in time series, and stores frame correlation images along the time series in the frame memory 306. A frame correlation image is generated by averaging of ultrasound images arranged in time series, the averaging including weighting associated with the time series. The frame correlation image generating unit 321A is realized by use of a CPU, various arithmetic operation circuits, or the like.

The edge intensity calculating unit 322A calculates an edge intensity of each frame correlation image. The edge intensity calculating unit 322A is realized by use of a CPU, various arithmetic operation circuits, or the like.

Based on the edge intensity calculated by the edge intensity calculating unit 322A, the frozen image selecting unit 310 selects a frozen image from the frame correlation images.

If images arranged in time series are displaced from each other due to movement of the ultrasound transducer 21, when a frame correlation image is generated therefrom, its edge intensity is reduced because the edges therein are blurred. Therefore, according to this reference example, since the frozen image selecting unit 310 selects a frozen image based on edge intensities, an image with less movement is able to be selected appropriately.

The frozen image selecting unit 310 may select a frozen image, based on amounts of specific frequency component from spatial frequencies calculated by Fourier transformation of frame correlation images, instead of the edge intensities.

Furthermore, similarly to the second embodiment and the third embodiment, ultrasound images may each be divided into plural areas, and an edge intensity or a spatial frequency may be calculated for each area.

Some examples of ultrasound endoscopes have been described above with respect to the embodiments, but the ultrasound observation device according to the disclosure may be applied to an external ultrasound probe that emits ultrasound from a body surface of a subject. The external ultrasound probe is normally used when an abdominal organ (a liver, gallbladder, or bladder), breasts (mammary glands, in particular), or a thyroid gland is/are observed.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound observation device configured to generate, based on ultrasound signals acquired by an ultrasound transducer performing transmission and reception to and from an observation target, plural ultrasound images along time series, the ultrasound observation device comprising:
a movement amount calculating circuit configured to calculate, from the plural ultrasound images, an amount of movement that is an amount moved by a subject captured in an ultrasound image of the latest frame relatively to the subject captured in an ultrasound image of a past frame;
a reliability determining circuit configured to determine a reliability of the amount of movement calculated by the movement amount calculating circuit; and
a frozen image selecting circuit configured to select a frozen image from the plural ultrasound images, based on the amount of movement and the reliability, when input of a freeze instruction signal has been received.

2. The ultrasound observation device according to claim 1, wherein
the ultrasound transducer is arranged at a distal end of an insertion portion to be inserted into a subject, and
the frozen image selecting circuit is configured to
calculate, based on the amount of movement, information related to movement in a direction parallel to a scan surface of the ultrasound transducer,
calculate, based on the reliability, information related to movement or rotation in a direction different from the direction parallel to the scan surface of the ultrasound transducer, and
select the frozen image.

3. The ultrasound observation device according to claim 1, wherein the frozen image selecting circuit is configured to select, as the frozen image, an image having more areas where the reliability is high, from the ultrasound images.

4. The ultrasound observation device according to claim 1, wherein the frozen image selecting circuit is configured to select the frozen image, based on distribution of the reliability in the ultrasound images.

5. The ultrasound observation device according to claim 1, wherein the frozen image selecting circuit is configured to select, as the frozen image, an ultrasound image high in the reliability in an area where a depth of the observation target relative to the ultrasound transducer is large.

6. The ultrasound observation device according to claim 1, wherein
the movement amount calculating circuit is configured to calculate a similarity between a measurement area set in the ultrasound image, and plural areas included in a predetermined area around the measurement area, and calculate the amount of movement by detecting an area high in the similarity, and
the reliability determining circuit is configured to determine the reliability, based on a distribution of the similarity calculated by the movement amount calculating circuit.

7. An operation method of an ultrasound observation device configured to generate, based on ultrasound signals acquired by an ultrasound transducer performing transmission and reception to and from an observation target, plural ultrasound images along time series, the operation method comprising:
calculating, from the plural ultrasound images, an amount of movement that is an amount moved by a subject captured in an ultrasound image of the latest frame relatively to the subject captured in an ultrasound image of a past frame;
determining a reliability of the calculated amount of movement; and
selecting a frozen image from the plural ultrasound images, based on the amount of movement and the reliability, when input of a freeze instruction signal has been received.
